(19) United States
(12) Patent Application Publication
(10) Pub. No.: US 2008/0242684 A1
(43) Pub. Date: Oct. 2, 2008

(54) METHODS OF ADMINISTRATION OF ADENOSINE A1 RECEPTOR ANTAGONISTS

(76) Inventor: Howard Dittrich, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/058,532

(22) Filed: Mar. 28, 2008

Related U.S. Application Data
(60) Provisional application No. 60/908,943, filed on Mar. 29, 2007.

(51) Int. Cl. A61K 31/522

(52) U.S. Cl. 514/263.34

(57) ABSTRACT

Provided herein are improved methods of treating subjects with adenosine A₁ receptor antagonists. The subject can be provided a composition that includes at least about 20-40 mg, preferably about 30 mg of an AA₁RA, such as KW-3902 or a pharmaceutically acceptable salt, prodrug, ester, amide, or metabolite thereof to said subject, while maintaining a plasma Cₘₐₓ of KW-3902 or its metabolites below about 600 nM, preferably below about 550 nM, following administration.

### RELATED APPLICATIONS

The present application claims priority to U.S. Provisional Application Ser. No. 60/908,943, filed on Mar. 29, 2007, by Dittrich, and entitled “IMPROVED METHODS OF ADMINISTRATION OF ADENOSINE A₁ RECEPTOR ANTAGONISTS,” the entire disclosure of which is herein incorporated by reference in its entirety.

### BACKGROUND OF THE INVENTION

1. Field of the Invention

The present invention relates to the field of medicine, and in particular to improved methods of using adenosine A₁ receptor antagonists to treat individuals in need thereof.

2. Description of the Related Art

Adenosine is involved in the regulation of renal haemodynamics, tubular reabsorption of fluid and solutes, and in renin release in kidneys. In contrast to other vascular beds, adenosine induces vasoconstriction in the kidney, thereby coupling renal perfusion to the metabolic rate of the organ. In addition to its renal and diuretic effects, adenosine modulates seizures. Seizures and convulsions are the consequence of temporary abnormal electrophysiologic phenomena of the brain, resulting in abnormal synchronization of electrical neuronal activity. Every individual has a seizure threshold, i.e., a tolerance point beyond which a seizure can be induced. For example, individuals who develop seizure disorders have a lower threshold for seizures than others. Sleep deprivation, prolonged or acute stress, exhaustion, fear, illness, increases in breathing rates or changes in blood sugar levels are exemplary factors known to lower the seizure threshold.

Adenosine exerts its biologic functions through binding to different G-Protein Coupled Receptors (“GPCRs”), e.g., A₁, A_{2A}, A_{2B}, A₃ and A₄. The adenosine A₁ receptor regulates renal fluid balance, as well as excitatory glutamatergic neurotransmission, which contributes to its anticonvulsant activity. Antagonists to A₁ receptors (AA₁RAs) cause diuresis and natriuresis without major changes in glomerular filtration rate (“GFR”) and decrease afferent arteriolar pressure. Xanthine-derived adenosine A₁ receptor antagonists, such as KW-3902, are effective diuretics, renal-protectants, and bronchodilators, also lower the seizure threshold of individuals.

The chemical name of the AA₁ RA KW-3902 is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

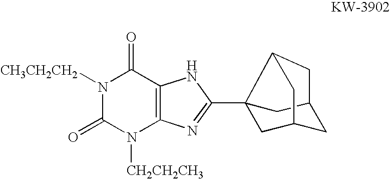

KW-3902 and related compounds are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

KW-3902 and related compounds have a diuretic effect, a renal-protecting effect, and a bronchiodilatory effect. Further, KW-3902, when combined with a standard diuretic is beneficial to subjects who are refractory to standard therapy. KW-3902 also blocks the tubuloglomerular feedback (“TGF”) mechanism mediated by adenosine (via A₁ receptors) described above. This ultimately allows for increased GFR and improved renal function, which results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, which can counteract the adverse effect of some diuretics, such as proximal diuretics, which active or promote TGF. See, e.g., U.S. Pat. No. 5,290,782, and U.S. patent application Ser. Nos: 10/830,617 filed Apr. 23, 2004, 11/248,479 filed Oct. 11, 2005, 11/248,905 filed Oct. 11, 2005, and 11/464,665, filed Jun. 16, 2006 the entire disclosure of all of which are hereby incorporated by reference herein, including any drawings.

There is a need for improved methods of administering KW-3902 and other AA₁RAs that retain the ability to function as effective adenosine A₁ receptor antagonists but that reduce or eliminate the undesirable central nervous system (CNS) side effects of adenosine A₁ receptor antagonism.

### SUMMARY OF THE INVENTION

Provided herein are improved methods of treating subjects with adenosine A₁ receptor antagonists. Some embodiments disclosed herein relate to methods of inducing diuresis, or maintaining or restoring the diuretic effect of a non adenosine-modifying diuretic in a subject. Other embodiments provide methods of maintaining, restoring, or improving renal function in a subject. Still other embodiments relate to methods of preventing or delaying the onset of renal impairment in subjects. Still other embodiments relate to methods of treating subjects suffering from congestive heart failure (CHF).

In some embodiments, the subject can be provided a composition that includes at least about 20-40 mg, preferably about 30 mg of an AA₁RA, such as KW-3902 or a pharmaceutically acceptable salt, prodrug, ester, amide, or metabolite thereof to said subject, while maintaining a plasma Cₘₐₓ of KW-3902 and its metabolites below about 600 nM, preferably below about 550 nM, following administration. In some embodiments, the AUC of KW-3902 and its metabolites is about 1000 to about 4000 nM. Preferably, the AA₁RA, such as KW-3902 is administered to maintain a Cₘᵢₙ above about 25 nM while maintaining a Cₘₐₓ below about 600 nM, preferably below about 550 nM, following administration.

In some embodiments, the subjects being treated can also have one of the following conditions: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

In some embodiments, the subject can be refractory to standard diuretic therapy. In other embodiments, the subject is not refractory to standard diuretic therapy. In some embodiments, the subject can have impaired renal function. In some embodiments, the subjects can have a decreasing creatinine clearance rate and/or elevated serum creatinine levels.

In some embodiments, the subject can have CHF, and can have impaired renal function. In some embodiments, the subjects with CHF can have a decreasing creatinine clearance rate and/or elevated serum creatinine levels. In some embodiments, the subject can have CHF and normal creatinine clearance and/or serum creatinine levels.

### BRIEF DESCRIPTION OF THE DRAWINGS

FIGS. 1A and 1B show the simulated serum concentrations of KW-3902 and the M1-trans metabolite of KW-3902 from single 25 mg or 30 mg intravenous doses of KW-3902 over time, respectively. The KW-3902 is administered over 2, 4, 6, 8, 12, or 24 hours, as indicated in the figure legend. Also shown in FIG. 1A is the simulated serum concentration of KW-3902 and M1-trans following a 10 mg dose of KW-3902 administered over two hours, followed by a 15 mg dose of KW-3902 over 4 hours (10/15), or a 15 mg dose of KW-3902 over two hours, followed by a 10 mg dose of KW-3902 over 4 hours (15/10). Also shown in FIG. 1B is the simulated serum concentration of KW-3902 and M1-trans following a 10 mg dose of KW-3902 over 2 hours followed by a 20 mg dose of KW-3902 over 4 hours (10/20); a 15 mg dose of KW-3902 administered over 2 hours followed by a 15 mg dose of KW-3902 over 4 hours (15/15), or a 20 mg dose of KW-3902 administered over 2 hours followed by a 10 mg dose of KW-3902 over 4 hours (20/10). The arrows in FIGS. 1A and 1B indicate the simulated concentration of the KW-3902 and the M1-trans metabolite combined following a single dose of 25 mg or 30 mg KW-3902, respectively, administered over the course of 4 hours.

FIGS. 2A-2D are graphs showing simulated pharmacodynamic characteristics of the indicated doses of KW-3902 administered by the indicated infusion rates. FIG. 2A shows the AUC of KW-3902 and the M1 trans metabolite following administration of the indicated amounts of KW-3902 over the indicated infusion times, FIG. 2B shows the Cₘₐₓ (nm) of KW-3902 and the M1-trans metabolite measured following administration of the indicated amounts of KW-3902 over the indicated infusion times. FIG. 2C shows the Cₘᵢₙ (nm) of KW-3902 and the M1-trans metabolite following administration of the indicated amounts of KW-3902 over the indicated infusion times, FIG. 2D shows the simulated concentration of M1-trans at 6 h hours following administration the indicated amounts of KW-3902 over the indicated infusion times.

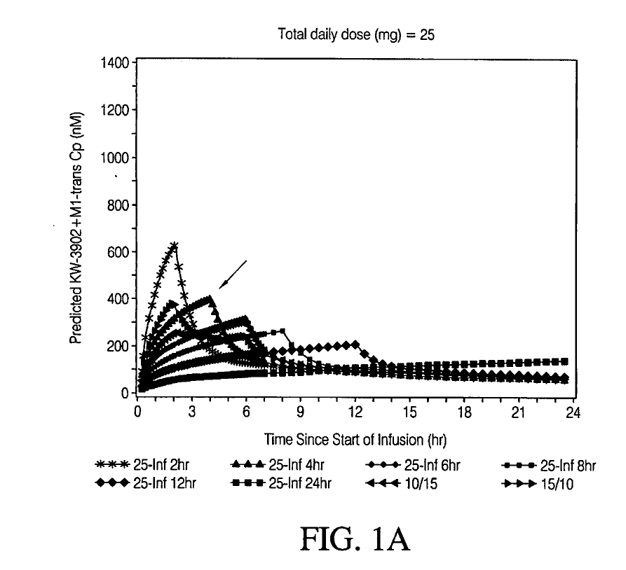

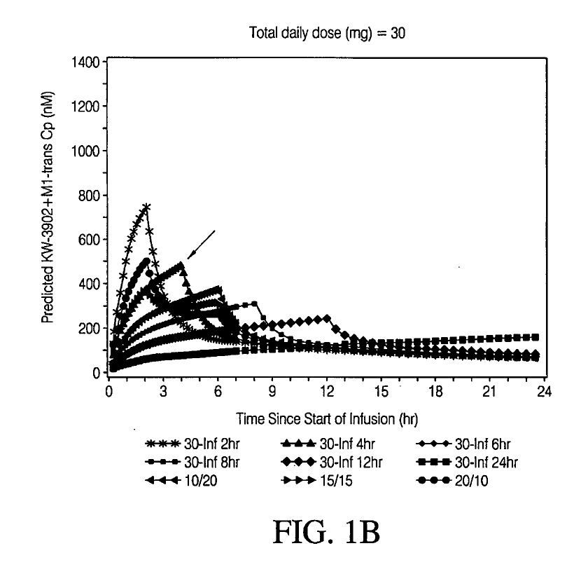

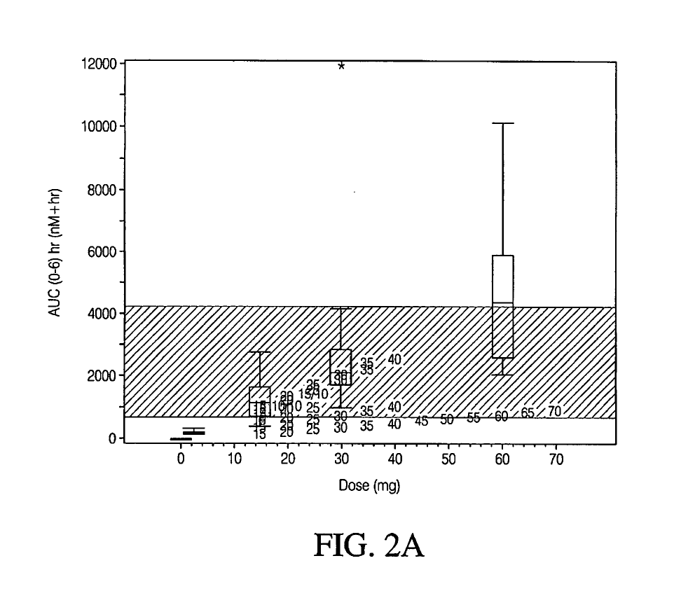

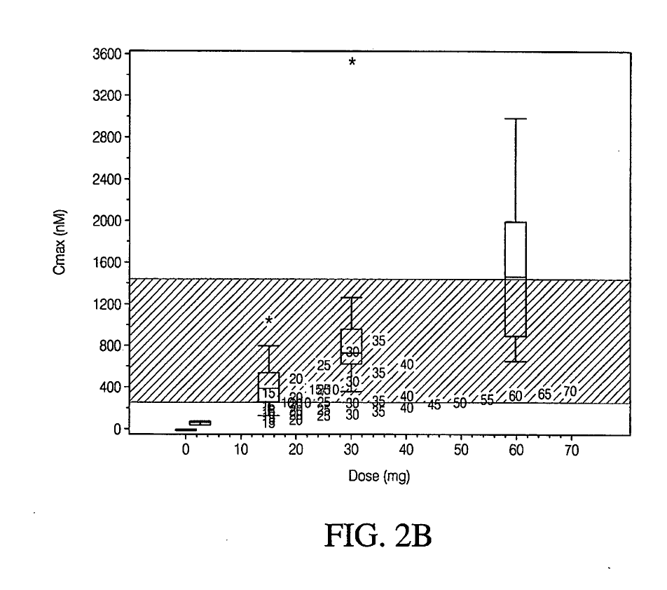

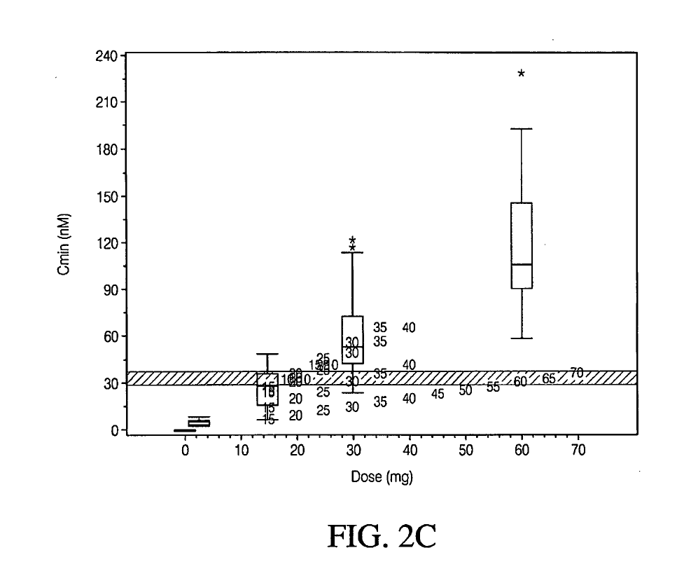

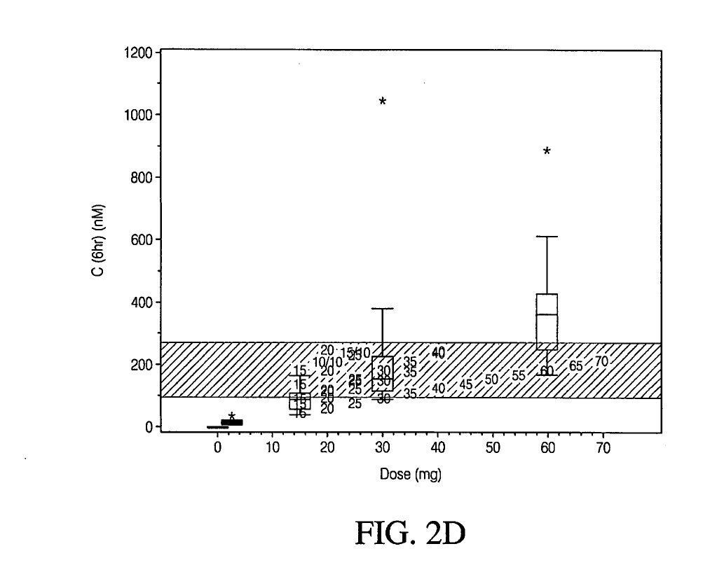

### DETAILED DESCRIPTION OF THE PREFERRED EMBODIMENT

A significant problem encountered in treating certain conditions with individual medications is that following a course of therapy the subjects become refractory to the treatment, i.e., the subjects begin to respond less and less to the medication until they do not respond at all. This problem is very common in subjects who suffer from, for example, cardiovascular disease, including individuals suffering from congestive heart failure (CHF) who are treated with diuretics.

Individual diuretics act on a specific segment of nephrons, e.g., proximal tubule, loop of Henle, or distal tubule. One mechanism by which diuretics increase urine volume is that they inhibit reabsorption of sodium and accompanying water passing through the nephron. Thus, for example, a loop diuretic inhibits reabsorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium reabsorption elsewhere in the nephron; the other is through feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

AA₁RAs act on the afferent arteriole of the kidney to produce vasodilation and thereby improve renal blood flow in subjects with CHF. They also block the TGF mechanism mediated by adenosine (via A₁ receptors) described above. This ultimately allows for increased GFR and improved renal function. In addition, AA₁RAs inhibit the reabsorption of sodium (and, therefore, water) in the proximal tubule, which results in diuresis.

AA₁RAs exert a diuretic effect by inhibiting the reabsorption of sodium in the proximal tubule of the nephron through adenosine A₁ receptors. In addition, AA₁RAs improve renal blood flow and glomerular filtration by inhibiting TGF, which is activated by diuretics that increase distal tubular sodium. Further, it appears that AA₁RAs have anti-oxidant properties in some conditions, such as radiographic contrast-mediated nephropathy, and therefore, may have similar properties in other conditions where oxygen-free radicals are injurious.

Administration of AA₁RAs to individuals in need of diuretics (e.g., presenting with congestive heart failure, diminished renal function, hypertension, asymptomatic left ventricular dysfunction, coronary artery disease, acute myocardial infarction, or suffering from a cardiovascular disease and in need of after-load reduction) may increase the probability of seizures. Not wishing to be bound by any particular mechanism or mode of action and offered only to expand the knowledge in the field, it is contemplated that the administration of AA₁RAs can decrease the seizure threshold in at-risk subjects.

Seizures and convulsions are the consequence of temporary abnormal electrophysiologic phenomena of the brain, resulting in abnormal synchronization of electrical neuronal activity. They can manifest as an alteration in mental state, tonic or clonic movements (discussed below) and various other symptoms. Tonic clonic seizures, also known as grand mal seizures involve two phases: a tonic phase and a clonic phase. The tonic phase involves vocalization, severe hyperextension (opisthotonos), possible respiratory arrest, cyanosis, and reflex bladder emptying. The clonic phase involves rhythmic generalized jerking, followed by prolonged unconsciousness.

Everyone individual has a seizure threshold, i.e., a tolerance point beyond which a seizure can be induced. For example, individuals who develop seizure disorders have a lower threshold for seizures than others. Sleep deprivation, prolonged or acute stress, exhaustion, fear, illness, increases in breathing rates or changes in blood sugar levels are exemplary factors known to lower the seizure threshold.

Accordingly, provided herein are improved methods of administration for AA₁RAs, such as KW-3902, that can minimize the risk of seizure and/or convulsions while maintaining desirable therapeutic effects.

### AA₁RAs

A number of AA₁RAs are known in the art, though currently, none are commercially available as therapeutics. AA₁RAs antagonize the A₁ receptor of adenosine selectively (e.g., they do not substantially antagonize other adenosine receptors). The majority of the known AA₁RAs are derivatives of xanthine and include compounds such as 1,3-dipropyl-8-{3-oxatricyclo[3.1.2.0.^{2,4}]oct-6(7)-yl}xanthine (also known as 1,3-dipropyl-8-[5,6-exo-epoxy-2(S)norbornyl]xanthine, ENX, CVT-124, and BG9928), 8-(3-noradamantyl)-1,3-dipropylxanthine (also known as KW-3902), theophyllilne, and caffeine. Other AA₁RAs are disclosed in U.S. Pat. Nos. 5,446,046, 5,631,260, and 5,668,139, the disclosures of which are all hereby incorporated by reference herein in their entirety, including any drawings. The scope of the present invention includes all those AA₁RAs now known and all those AA₁RAs to be discovered in the future.

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

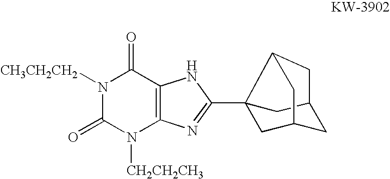

KW-3902 and related compounds useful in the practice of the embodiments described herein are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

In some embodiments, embodiments relate to improved methods of administering a xanthine-derivative compound of Formula I or a pharmaceutically acceptable salt thereof,

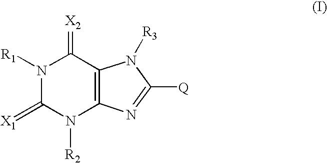

where

each of X₁ and X₂ independently represents oxygen or sulfur;

Q represents:

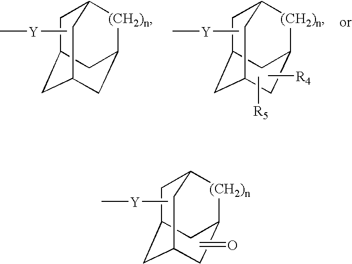

where Y represents a single bond or alkylene having 1 to 4 carbon atoms, n represents 0 or 1;

each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or

R₄ and R₅ are the same or different and each represent hydrogen or hydroxy, and when both R₄ and R₅ are hydrogen, at least one of R₁ and R₂ is hydroxy-substituted or oxo-substituted lower alkyl,

provided that when Q is

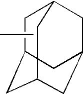

then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represents hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R₁ is propyl, 2-hydroxypropyl, 2-oxopropyl or 3-oxopropyl; R₂ is propyl, 2-hydroxypropyl or 3-hydroxypropyl.

In some embodiments Q is

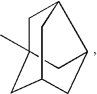

while in other embodiments Q is

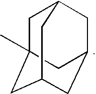

In other embodiments, Q is 9-hydroxy, 9-oxo or 6-hydroxy substituted 3-tricyclo[3.3.1.0^{3,7}]nonyl, or 3-hydroxy-1tricyclo[3.3.1.1^{3,7}]decyl.

In certain embodiments, the AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine (also referred to as “M1-trans”), 8-(cis-9-hydroxy-3-tricyclo[3.3.1.0^{3,7}]nonyl)-1,3-dipropylxanthine (also referred to as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof.

In other embodiments, the AA₁RA is a xanthine epoxide-derivative compound of Formula II or Formula III, or a pharmaceutically acceptable salt thereof,

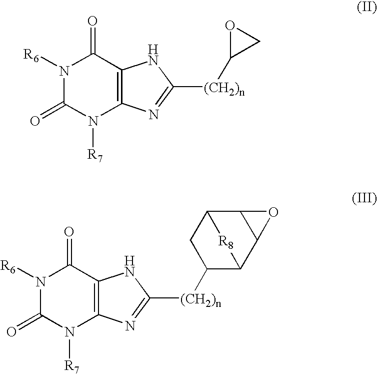

where R₆ and R₇ are the same or different, and can be hydrogen or an alkyl group of 1-4 carbons, R₈ is either oxygen or (CH₂)₁₋₄, and n=0-4.

The xanthine epoxide-derivative compound may be

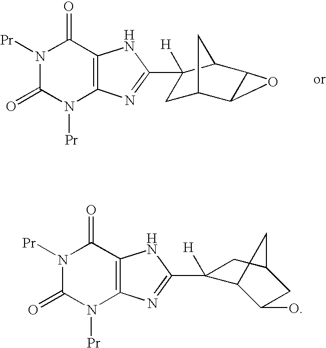

In some embodiments, the AA₁RA is KW-3902. KW-3902 and related compounds useful in the practice of the embodiments disclosed herein are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

Some embodiments provide improved methods of administering KW-3902 or pharmaceutically acceptable salts, esters, amides, metabolites, or prodrugs thereof.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which the AA₁RA is converted within the cells of a mammal. The pharmaceutical compositions described herein may include a metabolite of KW-3902, or any other AA₁RA instead of KW-3902 or the AA₁RA, respectively. The scope of the methods of the present invention includes those instances where an AA₁RA is administered to the subject, yet the metabolite is the bioactive entity.

Metabolites of KW-3902 are known. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In certain embodiments, the AA₁RA compound described herein such as KW-3902 is in a formulation described in U.S. Pat. No. 6,210,687, or U.S. Pat. No. 6,254,889, both or which are hereby incorporated by reference herein in their entirety, including any drawings.

### Administration of AA₁RAs

Provided herein are improved methods of treating subjects with AA₁RAs such as KW-3902 and the like. In some embodiments, the subject can be administered at least about 20 mg, at least about 25 mg, at least about 30 mg, at least about 35 mg, at least about 40 mg, or more of the AA₁RA (e.g., KW-3902) to provide a mean Cₘₐₓ of plasma levels of the AA₁RA and the metabolites thereof (e g., KW-3902 and M1-trans) below about 700 nM, below about 690 nM, below about 680 nM, below about 670 nM, below about 660 nM, below about 650 nM, below about 630 nM, below about 620 nM, below about 610 nM, below about 600 nM, below about 590 nM, below about 580 nM, below about 570 nM, below about 560, or below about 550 nM upon administration to the subject. Preferably, the administration of the AA₁RA (e.g., KW-3902) can provide at least about 25 mg to about 35 mg, e.g., for example about 30 mg of the combination of the of the AA₁RA and the metabolites thereof (e.g., KW-3902 and M1-trans) to the subject, while maintaining a Cₘₐₓ of below about 630 nM, e.g., below about 600 nM. In some embodiments, the administration of the AA₁RA (e.g., KW-3902) can provide at least about 20 mg of the AA₁RA (e.g., KW-3902) and its metabolites (e.g., KW-3902 and M1-trans) to the subject, while maintaining a Cₘₐₓ of below about 540 nM, e.g., below about 500 nM.

In some embodiments, the administration of the AA₁RA (e.g., KW-3902) can provide a Cₘᵢₙ above about 15 nM, about 16 nM, about 17 nM, about 18 nM, about 19 nM, about 20 nM, about 21 nM, about 22 nM, about 23 nM, about 24 nM, about 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, or 40 nM, while maintaining a Cₘₐₓ below about 700 nM, below about 690 nM, below about 680 nM, below about 670 nM, below about 660 nM, below about 650 nM, below about 630 nM, below about 620 nM, below about 610 nM, below about 600 nM, below about 590 nM, below about 580 nM, below about 570 nM, below about 560, or below about 550 nM following administration to the subject. Preferably, the administration of the AA₁RA (e.g., KW-3902) can provide a Cₘᵢₙ above about 20 nM, e.g., above about 25 nM, while maintaining a Cₘₐₓ below about of below about 630 nM, e.g., below about 600 nM following administration.

In some embodiments, the administration of the AA₁RA (e.g., KW-3902) can provide an AUC of about 1000 to about 4000 nM * hour, for example an AUC of about 1100 nM, 1200 nM, 1300 nM, 1400 nM, 1500 nM, 1500 nM, 1600 nM, 1700 nM, 1800 nM, 1900 nM, 2000 nM, 2100 nM, 2200 nM, 2300 nM, 2400 nM, 2500 nM, 2600 nM, 2700 nM, 2800 nM, 2900 nM, 3000 nM, 3100 nM, 3200 nM, 3300 nM, 3400 nM, 3500 nM, 3600 nM, 3700 nM, 3800 nM, 3900 nM, or about 4000 nM, or any number in between, * hr of the AA₁RA (e.g., KW-3902) and its metabolites (e.g., M1-trans).

In some embodiments, the AA₁RA (e.g., KW-3902) can be administered parenterally. For example, intramuscularly, subcutaneously, intravenously, via intramedullary injections, as well as intrathecal, direct intraventricular, intraperitoneal, intranasal, or intraocular injections or the like. Preferably, the AA₁RA (e.g., KW-3902) can provided intravenously in a continuous infusion.

In some embodiments, the AA₁RA (e.g., KW-3902) is provided in a single dose during the administration. For example, in some embodiments, about 20 mg, 30 mg, 40 mg or more of an AA₁RA (e.g., KW-3902) can be provided in a single dose, for example in a continuous intravenous infusion. In some embodiments, the AA₁RA (e.g., KW-3902) is provided in more than one dose during the administration, for example, two, three or more doses of the AA₁RA (e.g., KW-3902) can be provided in a single continuous intravenous infusion. Accordingly, in some embodiments, a dose of about 10 mg of an AA₁RA (e.g., KW-3902) can be provided followed by a dose of about 15 mg or 20 mg in a continuous infusion. Alternatively, a dose of about 15 mg or 20 mg of an AA₁RA (e.g., KW-3902) can be provided, followed by a dose of about 10 mg or 15 mg of the AA₁RA in a continuous infusion, and the like. Preferably, the continuous intravenous infusion provides a mean Cₘₐₓ of plasma AA₁RA levels (e.g., KW-3902) and its metabolites (e.g., M1-trans) below about 630 nM, e.g., below about 600 nM. Preferably, the continuous infusion provides a Cₘᵢₙ of the AA₁RA (e.g., KW-3902) and its metabolites (e.g., M1-trans) above about 20 nM, e.g., above about 25 nM.

In some embodiments, the AA₁RA (e.g., KW-3902) can be provided in a continuous infusion for a period of time of about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more. Preferably, the AA₁RA (e.g., KW-3902) can be provided in a continuous infusion for a period of time of about 3 hours, 3.5 hours, 4 hours, 4.5 hours, or 5 hours, 5.5 hours, 6 hours, or 6.5 hours, or any amount of time in between.

In some embodiments, a single dose of the AA₁RA (e.g., KW-3902) is provided in a continuous infusion over a period of about 3 hours, 3.5 hours, 4 hours or 4.5 hours, preferably about 4 hours. In some embodiments, two doses of the AA₁RA (e.g., KW-3902) can be provided in a continuous infusion over a period of about 4 hours, 4.5 hours, 5 hours, 5.5 hours, 6 hours, 6.5 hours, or 7 hours. Optionally, the first dose can be provided over about 1.5 to about 2.5 hours, preferably 2 hours, and the second dose can be provided over about 3.5 hours, 4 hours, or 4.5 hours, preferably about 4 hours.

In some embodiments, the AA₁RA (e.g., KW-3902) can administered via continuous intravenous infusion to provide a Cₘₐₓ that is below about 90%, below about 85%, below about 80%, or below about 75% of the Cₘₐₓ of the AA₁RA (e.g., KW-3902) and its metabolites (e.g., M1-trans) provided via continuous intravenous infusion over a period of two hours. Preferably, the AA₁RA also maintains a Cₘᵢₙ that is at least 80%, at least 90%, at least 95%, at least 96%, at least 97%, at least 98%, at least 99% or at least 100% as high as the Cₘᵢₙ when the AA₁RA (e.g., KW-3902) is provided via continuous intravenous infusion over a period of two hours.

In some embodiments, the AA₁RA (e.g., KW-3902) is administered orally. Preferably, the oral formulation provides mean Cₘₐₓ plasma AA₁RA levels and the metabolites thereof below about 700 nM, below about 690 nM, below about 680 nM, below about 670 nM, below about 660 nM, below about 650 nM, below about 630 nM, below about 620 nM, below about 610 nM, below about 600 nM, below about 590 nM, below about 580 nM, below about 570 nM, below about 560, or below about 550 nM upon administration to the subject. Preferably, the oral of the AA₁RA (e.g., KW-3902) can provide at least about 25 mg to about 35 mg, e.g., for example about 30 mg of the AA₁RA (e.g., KW-3902) and its metabolites (e.g., M1-trans) to the subject, while maintaining a Cₘₐₓ of below about 630 nM, e.g., below about 600 nM. Oral formulations with the desired pharmacokinetic profiles described herein can be created using methods known to those skilled in the art. A description of carrier materials useful in the oral formulations described herein can be found in the Remington: The Science and Practice of Pharmacy (20^{th} ed, Lippincott Williams & Wilkens Publishers (2003)), which is incorporated herein by reference in its entirety.

The present inventors have made the discovery that administration of an AA₁RA (e.g., KW-3902) provides for improved renal function in individuals receiving chronic diuretic therapy that persists over an unexpectedly long period of time.

Thus, in some embodiments, an individual receiving chronic diuretic therapy is administered doses of a therapeutically effective amount of AA₁RA about every four days to about every month. Accordingly, in some embodiments, the AA₁RA can be administered to the individual receiving chronic diuretic therapy at least about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 40 days, about every 50 days, or about every 60 days, or any number of days in between.

As used herein, the phrase “chronic diuretic therapy” or variations thereof, e.g., “chronic diuretics” refers to continuous diuretic therapy (e.g., at least daily therapy) for a period of time. Individuals identified as receiving chronic diuretic therapy, therefore, can refer to individuals that have been taking daily diuretics continuously over at least about three weeks, at least about 4 weeks, at least about 6 weeks, at least about 10 weeks, or at least about 12 weeks, or more, or for any period of time in between. Continuation of chronic diuretic therapy refers to substantially uninterrupted daily diuretic therapy.

Combinations of AA₁RAs with Other Therapeutics

In some embodiments provided herein, the subject to be treated by the methods described herein can be administered an AA₁RA (e.g., KW-3902) as described above in combination with another compound or therapeutic such as a non adenosine-modifying diuretic, an ACE, an ARB, a beta blocker, an aldosterone inhibitor, anticonvulsant or other compound or any combination thereof. In some embodiments, the administering step comprises administering said non adenosine-modifying diuretic, or other therapeutic (e.g., ACE, ARB, beta blocker, anticonvulsant and the like) and said AA₁RA nearly simultaneously. These embodiments include those in which the AA₁RA and the non adenosine-modifying diuretic, or other therapeutic (e.g., ACE, ARB, beta blocker, aldosterone inhibitor, anticonvulsant and the like) are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering the non adenosine-modifying diuretic, or other therapeutic (e.g., ACE, ARB, beta blocker, aldosterone inhibitor, anticonvulsant and the like) first and then administering the AA₁RA (e.g., KW-3902). In yet other embodiments, the administering step comprises administering the AA₁RA (e.g., KW-3902), first, and then administering the non adenosine-modifying diuretic, or other therapeutic (e.g., ACE, ARB, beta blocker, aldosterone inhibitor, anticonvulsant and the like). In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally. Preferably, in embodiments wherein the subject is administered an AA₁RA and an anticonvusant, the anticonvulsant is provided before, e.g. 5 minutes, 10 minutes, 15 minutes, 20 minutes, 30 minutes, 1 hour, 2 hours or more, the AA₁RA. In other preferred embodiments, the anticonvulsant and the AA₁RA are administered substantially simultaneously.

In some embodiments, the embodiments provided herein provide for the administration of an AA₁RA (e.g., KW-3902) as described above and a non-adenosine modifying diuretic. In some embodiments, the non-adenosine modifying diuretic is a proximal diuretic, i.e., a diuretic that principally acts on the proximal tubule. Examples of proximal diuretics include, but are not limited to, acetazolamide, methazolamide, and dichlorphenamide. Carbonic anhydrase inhibitors are known to be diuretics that act on the proximal tubule, and are therefore, proximal diuretics. Thus, some embodiments provide compositions that include the combination of an AA₁RA (e.g., KW-3902), with a carbonic anhydrase inhibitor. Combinations of an AA₁RA (e.g., KW-3902), with any proximal diuretic now known or later discovered are within the scope of the embodiments disclosed herein.

In other embodiments, the non-adenosine modifying diuretic is a loop diuretic, i.e., a diuretic that principally acts on the loop of Henle. Examples of loop diuretics include, but are not limited to, furosemide (LASIX®), bumetanide (BUMEX®), and torsemide (TOREM®). Combinations of an AA₁RA with any loop diuretic now known or later discovered are within the scope of the embodiments disclosed herein. In certain embodiments, the non adenosine-modifying diuretic used in the methods of the present invention is furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenously, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

In yet other embodiments, the non-adenosine modifying diuretic is a distal diuretic, i.e., a diuretic that principally acts on the distal nephron. Examples of distal diuretics include, but are not limited to, metolazone, thiazides and amiloride. Combinations of an AA₁RA with any distal diuretic now known or later discovered are within the scope of the embodiments disclosed herein.

In some embodiments, the subject can be administered an AA₁RA (e.g., KW-3902) as described and a beta-blocker. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The embodiments described herein include all beta-blockers now known and all beta-blockers discovered in the future.

In some embodiments provided herein, a subject is administered an AA₁RA (e.g., KW-3902) as described above and an angiotensin converting enzyme inhibitor or an angiotensin II receptor blocker. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The embodiments described herein include all ACE inhibitors now known and all ACE inhibitors discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. The embodiments disclosed herein include all ARBs now known and all ARBs discovered in the future.

In some embodiments provided herein, a subject is administered KW-3902 or a pharmaceutically acceptable salt, ester, amide, metabolite thereof and an aldosterone inhibitor. A number of aldosterone inhibitors are commercially available. These compounds include, but are not limited to, spironolactone (ALDACTONE®) and eplerenone (INSPRA®). The embodiments disclosed herein include all aldosterone inhibitors now known and all aldosterone inhibitors discovered in the future.

In some embodiments of the methods described herein, the subject can be provided an anticonvulsant, in addition to an AA₁RA. Several anticonvulsants are known in the art and are useful in the compositions and methods described herein. See, e.g., U.S. Patent Application Publication No. 2005/0070524. Extensive listings of anticonvulsants can also be found, e.g., in Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083, the disclosures of which are hereby expressly incorporated by reference in their entireties. Non-limiting examples of anticonvulsants that can be used in the compositions and methods disclosed herein include diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof. However, the inclusion of other anticonvulsants, now known or discovered in the future, is within the scope of the present invention.

The methods described herein can include providing to a subject an amount of an anticonvulsant that will be therapeutically or prophylactically effective in the treatment or control of seizures. It will be appreciated that the amount of anticonvulsant contained in an individual dose of each dosage form of the compositions need not in itself constitute an effective prophylactic amount, as the necessary effective amount could be reached by administration of a number of individual doses. Those skilled in the art will appreciate that the amount of anticonvulsant agent present in the compositions and administered to individuals disclosed herein will vary depending upon the age, sex, and bodyweight of the subject to be treated, the particular method and scheduling of administration, and what other anticonvulsant agent, if any is present in the compositions disclosed herein or administered in the methods disclosed herein. Dosage amounts for an individual patient may thus be above or below the typical dosage ranges. Generally speaking, the anticonvulsant agent can be employed in any amount known to be effective at treating, preventing or controlling seizures. The doses may be single doses or multiple doses per day, with the number of doses taken per day and the time allowed between doses varying depending on the individual needs of the patient. Optimization of treatment, including dosage amount, method and time of administration can be routinely determined by the skilled practitioner. Specific dosage levels for anticonvulsants that can be used in the pharmaceutical compositions and methods described herein, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of which are all hereby expressly incorporated by reference. Representative examples of dosage ranges of anticonvulsants are described below, however, it should be noted that the dosage ranges given below indicate only the typical dosage amounts administered to patients for that particular anticonvulsant agent for the treatment of seizures or epilepsy. Thus they should not be construed as limiting amounts for the purpose of the present invention, as actual therapeutically effective dosage amounts for a patient may be more or less than the exemplary dosage range, depending on the individual.

The following section describes in further detail several anticonvulsants useful in the methods described herein.

### Diazepam

By way of example, when the anticonvulsant agent is diazepam, it is typical when administered orally, the amount used is within the range of approximately 4 to 40 mg/day, usually in 2 to 4 doses. When administered parenterally, the amount used is typically within the range of approximately 5 to 30 mg. The dose may be repeated, but usually is not more than 600 mg/day.

### Midazolam

In another example, when the anticonvulsant agent is midazolam, it is typical that the amount used is within the range of approximately 0.07-0.08 mg/kg/day (approximately 5 mg).

Phenytion/Fosphenytion

By way of example, when the anticonvulsant agent is phenyloin or fosphenytion, it is typical that the amount used is within the range of approximately 200 to 600 mg/day. The initial dose is typically within the range of approximately 3 to 5 mg/kg (200 to 400 mg/day) in 2 to 3 divided doses if given orally or approximately 10 to 20 mg/kg in one dose if given parenterally.

### Phenobarbital

By way of example, when the anticonvulsant agent is Phenobarbital, it is typical that when administered orally, the amount used is within the range of approximately 30 to 320 mg/day. When administered parenterally, the amount used is typically within the range of approximately 100 to 320 mg/. The dose may be repeated, but usually is not more than 600 mg/day.

### Mysoline

In another example, when the anticonvulsant agent is mysoline, it is typical that the amount used is within the range of 100 to 125 mg/day, but usually not more than 2000 mg/day.

### Clonazepam

In another example, when the anticonvulsant agent is clonazepam, it is typically that the amount used is within the range of approximately 0.5 to 20 mg/day in 2 to 4 divided doses, with the initial dose typically being approximately 1.5 mg/day.

### Clorazepate

By way of example, when the anticonvulsant agent is clorazepate, it is typical that the amount used is within the range of approximately 15 to 90 mg/day in 1 to 4 divided doses, with the initial dose typically being approximately 7.5 to 22.5 mg/day.

### Carbamazepine

By way of example, when the anticonvulsant agent is carbamazepine, it is typical that the amount used is within the range of approximately 400 to 2400 mg/day divided into 2 to 4 doses, with the initial dose typically being approximately 100 to 200 mg taken 1 to 2 times per day. Optimal dosage amounts will vary depending on the needs of the individual patient, but preferably will not exceed 1200 mg/day. Dosages may be administered one to four times per day, depending on the needs of the individual patient and the dosage form. Typically, a low initial dose with a gradual increase to the minimum effective dose is advised.

### Oxcarbazepine

In a further example, when the anticonvulsant agent is oxcarbazepine, it is typical that the amount used is within the range of approximately 900 to 3000 mg/day with the initial dose typically being approximately 400 to 600 mg/day in two divided doses.

### Valproic Acid/Sodium Valproate

In a further example, when the anticonvulsant agent is valproic acid, sodium valproate, or derivatives thereof, dose can be based on body weight. Typically the amount used is within the range of approximately 10 to 60 mg/kg/day (or 375 to 4000 mg/day) given in 2 to 4 divided doses, with the initial dose typically being approximately 5 to 30 mg/kg/day (or 250 to 750 mg/day) given at 2 to 4 divided doses. The initial dose is then typically gradually increased by 5 to 10 mg/kg/week, as needed.

### Gabapentin

In a further example, when the anticonvulsant agent is gabapentin, it is typical that that amount used is within the range of approximately 600 to 4800 mg/day, with the initial does typically being approximately 300 to 900 mg/day. The total daily dose is typically divided, and administered in 3 to 4 doses per day.

### Topiramate

In another example, when the anticonvulsant agent is topiramate, it is typical that the amount used is within the range of approximately 200 to 400 mg/day, with the initial dose typically being approximately 25 to 50 mg/day and slowly adjusted upwards as needed. Doses are typically divided and administered twice daily.

### Felbamate

In a further example, when the anticonvulsant agent is felbamate, it is typical that the amount used is within the range of approximately 600 to 3600 mg/day in 3 to 4 divided doses, with the initial dose typically being approximately 600 to 1200 mg/day.

### Tiagabin

In a further example, when the anticonvulsant agent is tiagabin, it is typical that the amount used is within the range of approximately 32 to 64 mg/day, with the initial dose typically being approximately 4 mg/day. Doses are typically divided and given 2 to 4 times daily.

### Lamotrigine

In yet another example, when the anticonvulsant agent is lamotrigine, the amount administered will vary depending on patient age and what other anticonvulsant agents, if any, are co-administered. The amount of lamotrigine administered is typically within the range 25 mg every other day to 700 mg/day. The amount of lamotrigine administered to patient who are also taking enzyme-inducing anticonvulsant agents (e.g., carbamazepine, Phenobarbital, phenyloin, and/or primidone) but are not taking valproic acid (or derivatives) is preferably within the range of approximately 50 to 500 mg/day. The amount of lamotrigine administered to patients who are taking enzyme inducing anticonvulsant agents and valproic acid is preferably within the range of approximately 25 mg every other day to 400 mg/day. Initial doses are usually within the lower end of the dosage ranges, and can be increased slowly, as needed, to avoid side effects.

### Famotidine

In still another example, when the anticonvulsant agent is famotidine, it is typical that the amount administered is approximately 20 mg/day, or more, but does not typically exceed 640 mg/day.

### Ethotoin

In yet another example, when the anticonvulsant agent is ethotoin, it is typical that the amount used is within the range of approximately 125 to 250 milligrams 4 to 6 times a day. Typically, the dose usually does not exceed more than 3000 mg a day.

### Mephobarbital

In another example, when the anticonvulsant agent is mephobarbital, it is typical that the amount used is within the range of approximately 32-100 mg 3 to 4 times a day, or 200 to 600 mg day in 3 to 4 divided doses.

### Metharbital

In another example, when the anticonvulsant agent is metharbital, it is typical that the amount used is within the range of approximately 100-300 mg a day, in 1 to 3 divided doses. Typically, the dose usually does not exceed more than 800 mg/day.

### Ethosuximide

In yet another example, when the anticonvulsant agent is ethosuximide, it is typical that the amount used is within the range of approximately 500 to 2000 mg/day, with the initial dose typically being approximately 250 to 500 mg/day. The total daily dose may be administered in one daily dose, or divided and given in two doses per day.

### Methsuximide

In yet another example, when the anticonvulsant agent is methsuximide, it is typical that the amount used is within the range of approximately 300 to 1200 mg/day.

### Trimethadione/Paramethadione

In yet another example, when the anticonvulsant agent is trimethadione or paramethadione, it is typical that the amount used is within the range of approximately 300 mg 3 to 4 times per day. Typically, the daily dose will not exceed 2400 mg/day, divided in 3 to 4 doses.

### Phenacemide

In yet another example, when the anticonvulsant agent is Phenacemide, it is typical that the amount used is within the range of approximately 500 mg three times a day. Typically, the daily dose will not exceed 5000 mg/day.

### Acetozolamide

In yet another example, when the anticonvulsant agent is acetozolamide, it is typical that the amount used is within the range of approximately 10 mg/kg/day in 1-3 divided doses.

### Clobazam

In yet another example, when the anticonvulsant agent is clobazam, it is typical that the amount used is within the range of approximately 500 to 2000 mg/day, with the initial dose typically being approximately 250 to 500 mg/day. The total daily dose may be administered in one daily dose, or divided and given in two doses per day.

### Levetiracetam

In a further example, when the anticonvulsant agent is levetiractam, it is typical that the amount used is within the range of approximately 1000 to 3000 mg/day, with the initial dose typically being approximately 1000 mg/day in two divided doses.

### Primidone

In another example, when the anticonvulsant agent is primidone, it is typical that the amount used is within the range of approximately 250 to 2000 mg/day in divided doses, with the initial dose typically being approximately 100 to 125 mg/day.

### Lorazepam

In another example, when the anticonvulsant agent is lorazepam, it is typical that administration is by intravenous bolus injection, at approximately 0.07 mg/kg (to a maximum of 4 mg) is given, and this can be repeated once after 20 minutes if no effect has been observed.

Thiopentone

In a further example, when the anticonvulsant agent is thiopentone, it is typical that the amount used is 100-250 mg initially in a bolus injection, with further 50 mg boluses every 2-3 minutes until seizures are controlled. Intravenous infusion is then typically continued at between 3 and 5 mg/kg per hour, and at thiopentone blood levels of about 40 mg/l.

### Propofol

In a still further example, when the anticonvulsant agent is propofol, it is typical that the amount used is within the range of 1-2 mg/kg initially as a bolus dose, which can be repeated if seizures continue, succeeded by an infusion of 1-15 mg/kg per hour.

### Zonisamide

In another example, when the anticonvulsant agent is zonisamide, it is typical that the amount used is within the range of approximately 100 to 8000 mg/day in two divided doses, with the initial dose typically being approximately 100 mg/day in one dose.

In some embodiments, the composition can include more than one anticonvulsant and an AA₁RA. For example, the composition can include 2 or 3 or more anticonvulsant agents.

Methods of Treating Subjects with AA₁RAs

Some embodiments relate to methods of improving diuresis while maintaining renal function in a subject with fluid overload, by administering an AA₁RA (e.g., KW-3902) as described above.

Typically, renal function is measured by plasma concentrations of creatinine, urea, and electrolytes. Creatinine is a byproduct of normal muscle metabolism that is produced at a fairly constant rate in the body and normally filtered by the kidneys and excreted in the urine. It will be appreciated that any method known to those skilled in the art for measuring renal function can be used in the methods described herein. For example, serum creatinine levels, urine creatinine levels, glomerular filtration rate (GFR) and renal plasma flow (RPF) can be used to assess renal function.

In the context of the present disclosure, by maintaining renal function it is meant that the renal function, as measured by creatinine clearance rate, remains unchanged for a period of time after the start of the therapy. In other words, by “maintaining” renal function it is meant that the rate of renal impairment, i.e., the rate of decrease in the urine creatinine clearance rate, is slowed or arrested for a period of time.

Accordingly, in some embodiments, the subjects exhibit a GFR of less than about 80 mL/min, for example about 20 mL/min, 30 mL/min, 40 mL/min, 50 mL/min, 60 mL/min 70 mL/min or 75 mL/min, or any number in between. Accordingly, in some embodiments, the individual exhibits mildly impaired renal function (e.g., a GFR of about 50 to about 80 mL/min). In some embodiments, the individual exhibits moderately impaired renal function (e.g., a GFR of about 30 mL/min to about 50 mL/min). In yet other embodiments, the individual exhibits severely impaired renal function (e.g., a GFR of equal or less than about 30 mL/min). In some embodiments, the individuals suffering from renal impairment can exhibit a urine creatinine clearance rate of about 20 mg/dL to about 80 mg/dL, e.g., about 25 mg/dL, about 30 mg/dL, about 35 mg/dL, about 40 mg/dL, about 45 mg/dL, about 50 mg/dL, about 55 mg/dL, about 60 mg/dL, about 65 mg/dL, about 70 mg/dL, about 75 mg/dL, about 80 mg/dL, or more, or any number in between. In some embodiments, patients exhibit a GFR of less than about 80 mL/min, for example about 20 mL/min, 30 mL/min, 40 mL/min, 50 mL/min, 60 mL/min 70 mL/min or 75 mL/min, or any number in between.

In certain embodiments, the subject may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the subject is a human.

In some embodiments, the subject is identified as being “at risk” for developing a seizure or convulsions. For example, in some embodiments, the subject has been identified as having at least one of the following conditions: history of seizure, stroke within two years, brain tumor, brain surgery within two years, encephalitis within two years, history of penetrating head trauma closed head injury with loss of consciousness over 30 minutes within two years, history of alcohol withdrawal seizure, advanced Alzheimer's disease, or advanced multiple sclerosis.

Also provided herein are methods of maintaining or restoring the diuretic effect of a non adenosine-modifying diuretic in a subject. Diuretics are compounds that elevate the rate of bodily urine excretion (diuresis). Diuretics can also decrease the extracellular fluid (ECF) volume, and are primarily used to produce a negative extracellular fluid balance. Diuretics function by interfering with sodium and water re-absorption in the nephrons. In general, they increase the rate of sodium excretion from the body, thereby decreasing the volume of the ECF. The increase in sodium excretion restores salt homeostasis and lower tonicity, translating into lower blood pressure. The excretion of salt is usually accompanied by the loss of a proportional amount of water.

Individual diuretics act on a specific segment of nephrons, e.g., the proximal tubule, loop of Henle, or distal tubule. Thus, for example, a loop diuretic inhibits re-absorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium re-absorption elsewhere in the nephron; the other is to feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

Diuretics fall into four classes depending on their mode and locus of action: carbonic anhydrase inhibitors such as acetazolamide inhibit the absorption of NaHCO₃ and NaCl in the proximal tubule; loop diuretics such as furosemide act on the loop of Henle by inhibiting the Na⁺/K+/2Cl⁻ transporter; thiazide type diuretics which inhibit Na⁺/Cl⁻ co-transporters in the distal tubule; and potassium sparing diuretics act on the collecting duct, and decrease the sodium absorption while sparing K+ (i.e., as opposed to the other three categories that promote loss of potassium).

In preferred embodiments, the diuretic is non-adenosine modifying diuretic. In some embodiments, the non-adenosine modifying diuretic is a proximal diuretic, i.e., a diuretic that principally acts on the proximal tubule. Examples of proximal diuretics useful in the methods described herein include, but are not limited to, acetazolamide, methazolamide, dichlorphenamide, and carbonic anhydrase inhibitors.

In other embodiments, the non-adenosine modifying diuretic is a loop diuretic, i.e., a diuretic that principally acts on the loop of Henle. Examples of loop diuretics useful in the methods described herein include, but are not limited to, furosemide (LASIX®), bumetanide (BUMEX®), and torsemide (TOREM®).

In yet other embodiments, the non-adenosine modifying diuretic is a distal diuretic, i.e., a diuretic that principally acts on the distal nephron. Examples of distal diuretics useful in the methods described herein include, but are not limited to, metolazone, thiazides and amiloride.

In certain embodiments, the individual being treated by the methods of the present invention suffers from renal impairment. In other embodiments, the individual does not suffer from renal impairment. These individuals include those who suffer from heart failure, such as congestive heart failure, or other maladies that result in fluid overload, without having yet disrupted normal kidney function. In some embodiments, the individual being treated by the methods described herein is refractory to standard diuretic therapy. In other embodiments, the individual is not refractory to standard diuretic therapy.

Methods disclosed herein also relate to slowing or reversing an existing or developing renal impairment in a patient. In the context of the present disclosure, by “slowing” renal impairment it is meant that a decrease in renal function, as manifested for example by a decreasing creatinine clearance rate, is slowed or arrested for a period of time after the start of the therapy. In other words, by “slowing” renal impairment it is meant that the rate of renal impairment, i.e., the rate of decrease in the urine creatinine clearance rate, is slowed or arrested for a period of time. In the context of the present disclosure, by “reversing” an existing renal impairment it is meant that renal function is improved, as manifested for example by a cessation in a decreasing rate of creatine clearance, or an increase in the rate of urine creatinine clearance, for example.

Individuals with existing or developing renal impairment can exhibit a GFR of less than about 80 mL/min, for example about 20 mL/min, 30 mL/min, 40 mL/min, 50 mL/min, 60 mL/min 70 mL/min or 75 mL/min, or any number in between. Accordingly, in some embodiments, the individual exhibits mildly impaired renal function (e.g., a GFR of about 50 to about 80 mL/min). In some embodiments, the individual exhibit moderately impaired renal function (e.g., a GFR of about 30 mL/min to about 50 mL/min). In yet other embodiments, the individual exhibit severely impaired renal function (e.g., a GFR of equal or less than about 30 mL/min).

The methods described herein provide for the induction a diuretic effect in an animal, while reducing the potential of related adverse events occurring, such as seizures or convulsions. Accordingly, in another aspect, embodiments herein relate to methods of comprising identifying a subject in need thereof and administering to the subject an AA₁RA (e.g., KW-3902) as described above.

Other embodiments relate to methods of maintaining or restoring the diuretic effect of a non-adenosine modifying diuretic in a subject, comprising identifying a subject in need thereof, and administering to the subject an AA₁RA, (e.g., KW-3902), or a pharmaceutically acceptable salt, ester, amide, metabolite, or prodrug thereof, as described above in combination with a non-adenosine modifying diuretic. In some embodiments, the methods provided herein reduce the risk of the occurrence of an adverse event, such as a seizure. For example some embodiments relate to methods of maintaining or restoring the diuretic effect of a diuretic such as furosemide. In some embodiments, furosemide is administered in a dose of 20 mg, 40 mg, 60 mg, 80 mg, 100 mg, 120 mg, 140 mg, or 160 mg, or higher. As described above, the administration of the AA₁RA, furosemide, or both may be oral or intravenous. When furosemide is administered intravenously, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

Other embodiments relate to a method of maintaining or restoring renal function in a subject comprising identifying a subject in need thereof and administering an AA₁RA (e.g., KW-3902), or a pharmaceutically acceptable salt, ester, amide, metabolite, or prodrug thereof as described above, and administering and a second pharmaceutical composition capable of inducing a diuretic effect.

In the context of the present disclosure, by “maintaining” renal function it is meant that the renal function, as measured by creatinine clearance rate, remains unchanged for a period of time after the start of the therapy. In other words, by “maintaining” renal function it is meant that the rate of renal impairment, i.e., the rate of decrease in the creatinine clearance rate, is slowed or arrested for a period of time, however brief that period may be. By “restoring” renal function it is meant that the renal function, as measured by creatinine clearance rate, has improved, i.e., has become higher, after the start of the therapy.

In a further aspect, the present invention relates to a method of treating a subject with a pharmaceutical composition as described herein. In some embodiments, the subject is refractory to standard diuretic therapy.

Certain subjects who suffer from a cardiac condition, such as congestive heart failure, later develop renal impairment. The present inventors have discovered that if a subject presented with a cardiac condition, and little to no renal impairment, is treated with a pharmaceutical composition as described herein, the onset of renal impairment is delayed or arrested, compared to a subject who receives standard treatment. Thus, aspects of the present invention relate to a method of preventing the deterioration of renal function, delaying the onset of renal impairment, or arresting the progress of renal impairment in a subject comprising identifying a subject in need thereof, and administering a therapeutically effective amount of an AA₁RA (e.g., KW-3902), or a salt, ester, amide, metabolite, or prodrug thereof, and an anticonvulsant agent and a non-adenosine modifying diuretic.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the subject's overall feeling of well being or appearance. Treatment may also include lengthening the life of the subject, even if the symptoms are not alleviated, the disease conditions are not ameliorated, or the subject's overall feeling of well being is not improved. Thus, in the context of the present invention, increasing the urine output volume, decreasing the level of serum creatinine, or increasing creatinine clearance, may be considered treatment, even if the subject is not cured or does not generally feel better.

Still other embodiments disclosed herein provide methods of treating a subject suffering from CHF comprising identifying a subject in need thereof, and administering to said subject an AA₁RA (e.g., KW-3902), or a salt, ester, amide, metabolite, or prodrug thereof as described above and a non-adenosine modifying diuretic.

Also provided are embodiments that relate to a method of improving overall health outcomes, decreasing morbidity rates, or decreasing mortality rates in subjects comprising identifying a subject in need thereof, and administering to said subject an AA₁RA (e.g., KW-3902) as described above, or a salt, ester, amide, metabolite, or prodrug thereof, and administering and a non-adenosine modifying diuretic.

Overall health outcomes are determined by various means in the art. For example, improvements in morbidity and/or mortality rates, improvements in the subject's general feelings, improvements in the quality of life, improvements in the level of comfort at the end of life, and the like, are considered when overall health outcome are determined. Mortality rate is the number of subjects who die while undergoing a particular treatment for a period of time compared to the overall number of subjects undergoing the same or similar treatment over the same period of time. Morbidity rates are determined using various criteria, such as the frequency of hospital stays, the length of hospital stays, the frequency of visits to the doctor's office, the dosage of the medication being administered, and the like.

In yet another aspect, the methods of the present invention relate to the prevention of the deterioration of renal function in individuals comprising administering an AA1RA (e.g., KW-3902, or a salt, ester, amide, metabolite, or prodrug thereof as described above. In some embodiments, the method also includes that administration of a non-adenosine modifying diuretic.

In some embodiments, the subject whose overall health outcome, morbidity and/or mortality rate is being improved suffers from CHF. In other embodiments, the subject suffers from renal impairment.

Some embodiments disclosed herein are intended to provide treatment for cardiovascular disease, which may include congestive heart failure, hypertension, asymptomatic left ventricular dysfunction, coronary artery disease, or acute myocardial infarction. In some instances, subjects suffering from a cardiovascular disease are in need of after-load reduction. The methods disclosed herein are suitable to provide treatment for these subjects as well. Certain subjects who suffer from a cardiac condition, such as congestive heart failure, later develop renal impairment.

Other embodiments relate to the treatment of cardiovascular diseases using a combination of a beta-blocker, an AA₁RA, wherein the AA₁RA is administered as described above. The present inventors have discovered that the combination of AA₁RAs and beta blockers is beneficial in either congestive heart failure (CHF) or hypertension, or any of the other indications set forth herein. See, co-pending U.S. application Ser. No. 10/785,446 entitled “Method of Treatment of Disease Using and Adenosine A1 Receptor Antagonist,” filed Feb. 23, 2004, herein expressly incorporated by reference in its entirety.

Beta-blockers are known to have antihypertensive effects. While the exact mechanism of their action is unknown, possible mechanisms, such as reduction in cardiac output, reduction in plasma renin activity, and a central nervous system sympatholytic action, have been put forward. From various clinical studies, it is clear that administration of beta-blockers to subjects with hypertension results initially in a decrease in cardiac output, little immediate change in blood pressure, and an increase in calculated peripheral resistance. With continued administration, blood pressure decreases within a few days, cardiac output remains reduced, and peripheral resistance falls toward pretreatment levels. Plasma renin activity is also reduced markedly in subjects with hypertension, which will have an inhibitory action on the renin-angiotensin system, thus decreasing the after-load and allowing for more efficient forward function of the heart. The use of these compounds has been shown to increase survival rates among subjects suffering from CHF or hypertension. The compounds are now part of the standard of care for CHF and hypertension. The combination of an AA₁RA (e.g., KW-3902), a beta blocker, acts synergistically to further improve the condition of subjects with hypertension or CHF. The methods of administration provided herein can also reduce the potential of related adverse events occurring, such as seizures or convulsions. The diuretic effect of AA₁RAs, especially in salt-sensitive hypertensive subjects along with the blockage of beta adrenergic receptors decreases blood pressure through two different mechanisms, whose effects build on one another. In addition, most CHF subjects are also on additional diuretics. The combination allows for greater efficacy of other more distally acting diuretics by improving renal blood flow and renal function.

Beta-blockers are well established in the treatment of hypertension. The addition of AA₁RAs will further treat hypertension via its diuretic effect from inhibiting sodium reabsorption through the proximal tubule. In addition, since many hypertensive subjects are sodium sensitive, the addition of an AA₁RA to a beta-blocker will result in further blood pressure reduction. AA₁RA action on tubuloglomerular feedback further improves renal function to result in greater diuresis and lower blood pressure.

In another aspect, the invention relates to the treatment of renal and/or cardiac diseases using a combination of an AA₁RA administered as described above and an angiotensin converting enzyme (ACE) inhibitor or an angiotensin II receptor blocker (ARB). AA₁RAs, ACE inhibitors and ARBs have individually been shown to be somewhat effective in the treatment of cardiac disease, such as congestive heart failure, hypertension, asymptomatic left ventricular dysfunction, or acute myocardial infarction, or renal disease, such as diabetic nephropathy, contrast-mediated nephropathy, toxin-induced renal injury, or oxygen free-radical mediated nephropathy.

The present inventors have discovered that the combination of AA₁RAs and ACE inhibitors or ARBs is beneficial in either congestive heart failure (CHF) or hypertension. See, co-pending U.S. application Ser. No. 10/785,446. The use of ACE inhibitors and ARBs in CHF relies on inhibition of renin-angiotensin system. These compounds decrease the after-load, thereby allowing for more efficient forward function of the heart. In addition, renal function is “normalized” or improved such that subjects remove excess fluid more effectively. The use of these compounds has been shown to increase survival rates among subjects suffering from CHF or hypertension. The compounds are now part of the standard of care for CHF and hypertension.

The combination of AA₁RAs and ACE inhibitors or ARBs acts synergistically to further improve renal function for continued diuresis. In addition, most CHF subjects are also on additional diuretics. The combination allows for greater efficacy of other more distally acting diuretics by improving renal blood flow and renal function.

Both ACE inhibitors and ARBs are well established in the treatment of hypertension via their action through the renin-angiotensin system. The addition of AA₁RAs will further treat hypertension via its diuretic effect from inhibiting sodium reabsorption through the proximal tubule. In addition, since many hypertensive subjects are sodium sensitive, the addition of an AA₁RA to an ACE inhibitor or an ARB will result in further blood pressure reduction. AA₁RA action on tubuloglomerular feedback further improves renal function to result in greater diuresis and lower blood pressure.

ACE inhibitors and ARBs are also known to prevent some of the renal damage induced by the immunosuppressant, cyclosporin A. However, there is a renal damaging effect despite their use. The present inventors have discovered that the combination ACE inhibitors and ARBs with AA₁RAs would be more effective in preventing drug-induced nephrotoxicity, such as that induced by cyclosporin A, contrast medium (iodinated), and aminoglycoside antibiotics. In this setting there is renal vasoconstriction that can be minimized by both compounds. In addition, direct negative effects on the tubular epithelium by cyclosporin is less prominent in the setting of adenosine A₁ receptor antagonism, in that blocking A₁ receptors decreases active processes. Furthermore, there are fewer oxidative by-products that are injurious to the tubular epithelium. In addition, the inhibitory effect of AA₁RA blockade on the tubuloglomerular feedback mechanism helps preserve function in the setting of nephrotoxic drugs.

It is known that ACE inhibitors and ARBs are beneficial in preventing the worsening of renal dysfunction in diabetics as measured by albuminuria (proteinuria). Once diabetes begins, glucosuria develops and the kidneys begin to actively reabsorb glucose, especially through the proximal convoluted tubule. This active process may result in oxidative stress and begin the disease process of diabetic nephropathy. Early manifestations of this process are hypertrophy and hyperplasia of the kidney. Ultimately, the kidney begins to manifest other signs such as microalbuminuria and decreased function. It is postulated that the active reabsorption of glucose is mediated in part by adenosine A₁ receptors. Blockade of this process by an AA₁RA limits or prevents the early damage manifested in diabetics.

The combination of AA₁RA and ACE inhibitors or ARBs, as disclosed herein, works to limit both early and subsequent damage to the kidneys in diabetes. The presently disclosed combinations are given at the time of diagnosis of diabetes or as soon as glycosuria is detected in at risk subjects (metabolic syndrome). The long-term treatment using the combinations of the present invention includes daily administration of the pharmaceutical compositions described herein.

Other embodiments relate to a method of treating cardiovascular disease or renal disease comprising identifying a subject in need of such treatment, and administering a combination of an AA₁RA as described above, and an angiotensin converting enzyme (ACE) inhibitor or an angiotensin II receptor blocker (ARB) to said subject. In certain embodiments, the subject may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the subject is a human.

The methods disclosed herein are intended to provide treatment for cardiovascular disease, which may include congestive heart failure, hypertension, asymptomatic left ventricular dysfunction, or acute myocardial infarction. The methods disclosed herein can reduce the risk of adverse side effects, such as convulsions or seizures. In some instances, subjects suffering from a cardiovascular disease are in need of after-load reduction. The methods disclosed herein are suitable to provide treatment for these subjects as well.

The methods disclosed herein are also intended to provide treatment for renal disease, which may include renal hypertrophy, renal hyperplasia, microproteinuria, proteinuria, diabetic nephropathy, contrast-mediated nephropathy, toxin-induced renal injury, or oxygen free-radical mediated nephropathyhypertensive nephropathy, diabetic nephropathy, contrast-mediated nephropathy, toxin-induced renal injury, or oxygen free-radical mediated nephropathy.

Still other embodiments relate to methods of treating alkosis using an AA₁RA and anticonvulsant. Alkalosis is an acid-base disturbance caused by an elevation in plasma bicarbonate (HCO₃⁻) concentration. It is a primary pathophysiologic event characterized by the gain of bicarbonate or the loss of nonvolatile acid from extracellular fluid. The kidney preserves normal acid-base balance by two mechanisms: bicarbonate reclamation, mainly in the proximal tubule, and bicarbonate generation, predominantly in the distal nephron. Bicarbonate reclamation is mediated mainly by a Na⁺—H⁺ antiporter and to a smaller extent by the H⁺-ATPase (adenosine triphosphatase). The principal factors affecting HCO₃⁻reabsorption include effective arterial blood volume, glomerular filtration rate, potassium, and partial pressure of carbon dioxide. Bicarbonate regeneration is primarily affected by distal Na⁺ delivery and reabsorption, aldosterone, systemic pH, ammonium excretion, and excretion of titratable acid.

There are a number of different types of alkalosis, for instance metabolic alkalosis and respiratory alkalosis. Respiratory alkalosis is a condition that affects mountain climbers in high altitude situations.

To generate metabolic alkalosis, either a gain of base or a loss of acid must occur. The loss of acid may be via the upper gastrointestinal tract or via the kidney. Excess base may be gained by oral or parenteral HCO₃⁻ administration or by lactate, acetate, or citrate administration.

Factors that help maintain metabolic alkalosis include decreased glomerular filtration rate, volume contraction, hypokalemia, and aldosterone excess. Clinical states associated with metabolic alkalosis are vomiting, mineralocorticoid excess, the adrenogenital syndrome, licorice ingestion, diuretic administration, and Bartter's and Gitelman's syndromes.

The two types of metabolic alkalosis (i.e., chloride-responsive, chloride-resistant) are classified based upon the amount of chloride in the urine. Chloride-responsive metabolic alkalosis involves urine chloride levels less than 10 mEq/L, and it is characterized by decreased extracellular fluid (ECF) volume and low serum chloride such as occurs with vomiting. This type responds to administration of chloride salt. Chloride-resistant metabolic alkalosis involves urine chloride levels more than 20 mEq/L, and it is characterized by increased ECF volume. As the name implies, this type resists administration of chloride salt. Ingestion of excessive oral alkali (usually milk plus calcium carbonate) and alkalosis complicating primary hyperaldosteronism are examples of chloride resistant alkalosis.

Many subjects with edematous states are treated with diuretics. Unfortunately, with continued therapy, the subject's bicarbonate level increases and progressive alkalosis may ensue. Diuretics cause metabolic alkalosis by several mechanisms, including (1) acute contraction of the extracellular fluid (ECF) volume (NaCl excretion without HCO₃⁻), thereby increasing the concentration of HCO₃⁻ in the ECF; (2) diuretic-induced potassium and chloride depletion; and (3) secondary aldosteronism. Continued use of the diuretic or either of the latter two factors will maintain the alkalosis.

The addition of an AA₁RA allows continued diuresis and maintained renal function without worsening the alkalosis. The AA₁RA inhibits the active resorption of HCO₃⁻ across the proximal tubule of the kidney.

Thus, embodiments disclosed herein relate to a method of treating metabolic alkalosis, comprising identifying a subject in need thereof and administering a an adenosine A₁ receptor antagonist (AA₁RA) to said subject as disclosed herein. The methods above can reduce the potential of related adverse events occurring, such as seizures or convulsions In certain embodiments, the subject is suffering from high altitude mountain sickness. In some embodiments, the subject has edema. In some of these embodiments, the subject may be on diuretic therapy. The diuretic may be a loop diuretic, proximal diuretic, or distal diuretic. In other embodiments, the subject suffers from acid loss through the subject's upper gastrointestinal tract, for example, through excessive vomiting. In still other embodiments the subject has ingested excessive oral alkali. The methods of the present invention can be practiced with any compound that antagonizes adenosine A₁ receptors.

Still other embodiments relate to the treatment of diabetic neuropathy with an AA₁RA administered as described herein. The methods disclosed herein can reduce the potential of related adverse events occurring, such as seizures or convulsions. Uncontrolled diabetes causes damage to many tissues of the body. Kidney damage caused by diabetes most often involves thickening and hardening (sclerosis) of the internal kidney structures, particularly the glomerulus (kidney membrane). Kimmelstiel-Wilson disease is the unique microscopic characteristic of diabetic nephropathy in which sclerosis of the glomeruli is accompanied by nodular deposits of hyaline.

The glomeruli are the site where blood is filtered and urine is formed. They act as a selective membrane, allowing some substances to be excreted in the urine and other substances to remain in the body. As diabetic nephropathy progresses, increasing numbers of glomeruli are destroyed, resulting in impaired kidney functioning. Filtration slows and protein, namely albumin, which is normally retained in the body, may leak in the urine. Albumin may appear in the urine for 5 to 10 years before other symptoms develop. Hypertension often accompanies diabetic nephropathy.

Diabetic nephropathy may eventually lead to the nephrotic syndrome (a group of symptoms characterized by excessive loss of protein in the urine) and chronic renal failure. The disorder continues to progress, with end-stage renal disease developing, usually within 2 to 6 years after the appearance of renal insufficiency with proteinuria.

The mechanism that causes diabetic nephropathy is unknown. It may be caused by inappropriate incorporation of glucose molecules into the structures of the basement membrane and the tissues of the glomerulus. Hyperfiltration (increased urine production) associated with high blood sugar levels may be an additional mechanism of disease development.

The diabetic nephropathy is the most common cause of chronic renal failure and end stage renal disease in the United States. About 40% of people with insulin-dependent diabetes will eventually develop end-stage renal disease. 80% of people with diabetic nephropathy as a result of insulin-dependent diabetes mellitus (IDDM) have had this diabetes for 18 or more years. At least 20% of people with non-insulin-dependent diabetes mellitus (NIDDM) will develop diabetic nephropathy, but the time course of development of the disorder is much more variable than in IDDM. The risk is related to the control of the blood-glucose levels. Risk is higher if glucose is poorly controlled than if the glucose level is well controlled.

Diabetic nephropathy is generally accompanied by other diabetic complications including hypertension, retinopathy, and vascular (blood vessel) changes, although these may not be obvious during the early stages of nephropathy. Nephropathy may be present for many years before nephrotic syndrome or chronic renal failure develops. Nephropathy is often diagnosed when routine urinalysis shows protein in the urine.

Current treatments for diabetic nephropathy include administration of angiotensin converting enzyme inhibitors (ACE Inhibitors) during the more advanced stages of the disease. Currently there is no treatment in the earlier stages of the disease since ACE inhibitors may not be effective when the disease is symptom-free (i.e., when the subject only shows proteinuria).

Although the mechanism implicated in early renal disease in diabetics is that of hyperglycemia, a potential mechanism may be related to the active reabsorption of glucose in the proximal tubule. This reabsorption is dependent in part on adenosine A₁ receptors.

AA₁RAs act on the afferent arteriole of the kidney to produce vasodilation and thereby improve renal blood flow in subjects with diabetes. This ultimately allows for increased GFR and improved renal function. In addition, AA₁RAs inhibit the reabsorption of glucose in the proximal tubule in subjects with newly diagnosed diabetic mellitus or in subjects at risk for the condition (metabolic syndrome).

Thus, provided herein are embodiments that relate to a method of treating diabetic nephropathy while reducing the potential of related adverse events occurring, such as seizures or convulsions, comprising identifying a subject in need thereof and administering a an adenosine A₁ receptor antagonist (AA₁RA) to said subject as described herein. In certain embodiments the subject is pre-diabetic, whereas in other embodiments the subject is in early stage diabetes. In some embodiments the subject suffers from insulin-dependent diabetes mellitus (IDDM), whereas in other embodiments the subject suffers from non-insulin-dependent diabetes mellitus (NIDDM).

In certain embodiments, the methods of the present invention are used to prevent or reverse renal hypertrophy. In other embodiments, the methods of the present invention are used to prevent or reverse renal hyperplasia. In still other embodiments, the

Before people develop type II diabetes, i.e., NIDDM, they almost always have “pre-diabetes.” Pre-diabetic subjects have blood glucose levels that are higher than normal but not yet high enough to be diagnosed as diabetes. For instance, the blood glucose level of pre-diabetic subjects is between 110-126 mg/dL, using the fasting plasma glucose test (FPG), or between 140-200 mg/dL using the oral glucose tolerance test (OGTT). Blood glucose levels below 110 or 140, using FPG or OGTT, respectively, is considered normal, whereas individuals with blood glucose levels higher than 126 or 200, using FPG or OGTT, respectively, are considered diabetic. The methods of the present invention can be practiced with any compound that antagonizes adenosine A₁ receptors.

In certain aspects, the methods disclosed herein can be practiced using a combination therapy, i.e., where the AA₁RA is administered to the subject as described herein in combination with a second compound. In certain embodiments the second compound may be selected from a protein kinase C inhibitor, an inhibitor of tissue proliferation, an antioxidant, an inhibitor of glycosylation, and an endothelin B receptor inhibitor.

### Pharmaceutical Compositions

In some embodiments, the AA₁RA is administered in a pharmaceutical composition. The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions or lipid emulsions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Furthermore, the formulations of the present invention may be coated with enteric polymers. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, and U.S. Patent Application No. 60/674,080, the disclosures of which are each hereby incorporated by reference in their entirety, including any drawings.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the subject's condition. (See e.g., Fingl et al. 1975, in “The Pharmacological Basis of Therapeutics”, Ch. 1 p. 1). Typically, the dose range of the composition administered to the subject can be from about 0.5 to 1000 mg/kg of the subject's body weight. The dosage may be a single one or a series of two or more given in the course of one or more days, as is needed by the subject.

The daily dosage regimen for an adult human subject may be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### EXAMPLES

### Example 1

### Treatment of Individuals with Fluid Overload and Renal Impairment

Subjects presenting with New York Heart Association Class II-IV CHF and having an estimated creatinine clearance between 20 mL/min and 80 mL/min are identified. The subjects are taking an oral loop diuretic. The subjects can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects).

The subjects' medical history, physical examination, classification of CHF, vital signs, body weight, CHF signs and symptom scores, Holter monitor recording, chest X-ray, CBC chemistries, creatinine clearance, fluid intake, and urine output are determined on pre-treatment days −2 to −1, days 1 to 3 of the Treatment Period, day 4/early Termination and a follow up contact at day 30.

On treatment days, subjects receive an intravenous composition comprising between 20 and 40 mg of KW-3902, preferably about 30 mg, or a pharmaceutically acceptable salt, ester, amide, metabolite, or prodrug thereof intravenously over between 3 and 5 and preferably over about 4 hours vs. placebo as both monotherapy and concomitant therapy with diuretics on days 1 through 3. On day 1, the composition comprising KW-3902 (or placebo) is administered as a monotherapy. 6 hours after administration of KW-3902, IV loop diuretic is given to all treatment groups as needed. On days 2 and 3 KW-3902 derivatives are administered as combination therapy with intravenous furosemide, if clinically indicated. Final laboratory data are collected on day 4 or early termination. Follow-up phone contact is conducted on day 30.

Individuals receiving KW-3902 exhibit an improvement in kidney function as measured by serum creatinine levels compared to the baseline levels. This effect is grater than the effect seen in individuals who receive placebo. The combination of KW-3902 and non adenosine-modifying diuretics such as furosemide has a synergistic beneficial effect on diuresis, as measured by urine output. Individuals receiving KW-3902 derivatives of also require less non adenosine-modifying diuretic compared to individuals receiving placebo. At risk subjects do not experience seizures throughout the treatment period.

### Example 2

### Treatment of Individuals with Fluid Overload and Renal Impairment

A subject with fluid overload, as manifested by peripheral edema, dyspnea, and/or other signs or symptoms presents to the hospital, clinic, or doctor's office. The subject also shows some degree of renal impairment. The subjects can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects).

In addition to standard of care therapy which would include IV diuretics, e.g., IV furosemide, bumetanide and/or oral metolazone, the subject is also given a dose of about 20 mg to about 40 mg of KW-3902, preferably 30 mg, in injectable form over between 3 and 5, and preferably about 4 hours. The subject can be provided another dose of KW-3902 over 4 hours, and 40 mg of furosemide at 24 hour intervals or more or less frequently as needed. The subject's fluid intake and output, urine volume, serum and urine creatinine levels, electrolytes and cardiac function are monitored.

At the discretion of the attending physician, the dosage of furosemide can be increased to 60 mg, 80 mg, 100 mg, 120 mg, 140 mg, or 160 mg either during the treatment or as the initial dose, or furosemide can be given as a continuous infusion. The subjects

### Example 3

### Treatment of Individuals Refractory to Standard IV Diuretic Therapy

Subjects presenting with congestive heart failure having an estimated creatinine clearance between 20 mL/min and 80 mL/min and who are refractory to high dose diuretic therapy are identified and randomized to treatment groups receiving an intravenous infusion of between about 20 mg and about 40 mg KW-3902, and preferably about 30 mg, over between about 3 hours to about 5 hours, and preferably over 4 hours. The subjects can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects). Changes in urine output are measured hourly. The subjects' creatinine clearance rate is measured every 3 hours.

Subjects receiving KW-3902 therapy exhibit increased hourly urine volume over the ensuing 9 hours compared to subjects that receive placebo. Subjects administered KW-3902 also exhibit an improvement in creatinine clearance compared to subject that receive placebo.

A hospitalized subject who has been treated with maximum amounts of IV diuretic and is still symptomatic, fluid overloaded, or whose urine output is less than fluid intake is evaluated for further treatment. A dose of between about 20 mg and about 40 mg, preferably about 30 mg of KW-3902 in injectable form is infused through the IV line over the course of about 3 to about 5 hours, and preferably over the course of 4 hours. The subject receives continued treatment with furosemide. The subject's fluid intake and output, urine volume, serum and urine creatinine levels, electrolytes and cardiac function are monitored. Subjects do not experience seizures during the course of treatment.

At the discretion of the attending physician, the dosage of the KW-3902 derivative can be increased or decreased either during the treatment or as the initial dose, or furosemide can be given as a continuous infusion.

### Example 4

### Treatment of Individuals Refractory to Standard IV Diuretic Therapy

A hospitalized subject who has been treated with maximum amounts of IV diuretic and is still symptomatic, fluid overloaded, or whose urine output is less than fluid intake is evaluated for further treatment. A dose of a KW-3902 between about 20 mg and about 40 mg, and preferably about 30 mg injectable form is infused through the IV line over the course of about 3 hours to about 5 hours, and preferably over the course of about 4 hours. The subject receives continued treatment with furosemide, and also receives doses of KW-3902 derivatives of Formula (I), (II), (III), (IV), (V), or (VI) at 6 hour intervals, or more or less frequently as needed. The subject's fluid intake and output, urine volume, serum and urine creatinine levels, electrolytes and cardiac function are monitored.

At the discretion of the attending physician, the dosage of KW-3902 derivatives can be increased or decreased either during the treatment or as the initial dose, or furosemide can be given as a continuous infusion. The subjects show improved diuresis and do not experience seizures.

### Example 5

### Treatment of Individuals with Fluid Overload and Impaired Renal Function

A subject with fluid overload, as manifested by peripheral edema, dyspnea, and/or other signs or symptoms presents himself to the physician's office or clinic. The subject has been on a therapy regimen that includes oral diuretics and, in addition, to needing a higher dose of diuretics to manage his/her fluid balance, the subject is now showing impaired renal function. The subjects can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects).

The subject is administered a dose of about 20 mg to about 40 mg KW-3902 intravenously over about 3 to about 5 hours, preferably over the course of about 4 hours, concurrent with other diuretic therapy. The subject's fluid intake and output, urine volume, serum and urine creatinine levels, electrolytes and cardiac function are monitored.

At the discretion of the attending physician, the dosage of the KW-3902 derivative can be increased or decreased either during the treatment or as the initial dose. In addition, the dosage of furosemide can be increased to 60 mg, 80 mg, 100 mg, 120 mg, 140 mg, or 160 mg either during the treatment or as the initial dose. The subjects show improved diuresis and creatinine clearance, and do no experience seizures throughout the course of therapy.

### Example 7

### Treatment of Individuals with Fluid Overload

A subject with fluid overload, as manifested by peripheral edema, dyspnea, and/or other signs or symptoms presents to the physician's office or clinic. The subject has been on a therapy regimen that includes oral diuretics and needs a higher dose of diuretics to manage his/her fluid balance. To delay or prevent the onset of renal impairment and/or to delay the need to use higher dosages of standard diuretics, the subject is administered a dose of about 20 mg to about 40 mg, and preferably a dose of about 30 mg, KW-3902 IV, administered over the course of about 3 to about 5 hours, and preferably over the course of about 4 hours. The subject also receives concurrent with their diuretic therapy. The subject's fluid intake and output, urine volume, serum and urine creatinine levels, electrolytes and cardiac function are monitored.

At the discretion of the attending physician, the dosage of KW-3902 can be increased or decreased either during the treatment or as the initial dose. In addition, the dosage of furosemide can be increased to 60 mg, 80 mg, 100 mg, 120 mg, 140 mg, or 160 mg either during the treatment or as the initial dose. The subjects exhibit improved diuresis, their renal function is maintained, and the subjects do not experience a seizure throughout the course of treatment.

### Example 8

### Treatment of Individuals with Congestive Heart Failure

A subject with congestive heart failure presents to the physician's office or clinic. The subject can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects).

The subject is put on a therapy regimen that includes oral diuretics to manage his/her fluid balance. To delay or prevent the onset of renal impairment and/or to delay the need to use higher dosages of standard diuretics, the subject is also administered a dose of about 20 mg to about 40 mg KW-3902, preferably about 30 mg KW-3902, intravenously over the course of about 3 to about 5 hours, and preferably over the course of about 4 hours, concurrent with their diuretic therapy. The subject's fluid levels, urine volume, serum and urine creatinine levels, electrolytes and cardiac function are monitored.

At the discretion of the attending physician, the dosage of the KW-3902 derivative can be increased or decreased either during the treatment or as the initial dose. In addition, the dosage of furosemide can be increased to 60 mg, 80 mg, 100 mg, 120 mg, 140 mg, or 160 mg either during the treatment or as the initial dose.

### Example 9

### Improving Health Outcomes for of Individuals with Congestive Heart Failure

A subject with congestive heart failure presents to the physician's office or clinic. The subjects can also present with a one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis (“at risk” subjects).

The subject is put on a therapy regimen that includes oral diuretics to manage his/her fluid balance. To improve overall health outcomes (i.e., morbidity or mortality rates due to CHF), the subject is also provided a dose of about 20 mg to about 40 mg KW-3902, and preferably about 30 mg KW-3902 intravenously over the course of about 3 hours to about 5 hours, and preferably over the course of about 4 hours. The subject is provided their diuretic therapy concurrently. The subject's fluid levels, urine volume, serum and urine creatinine levels, electrolytes and cardiac function are monitored.

At the discretion of the attending physician, the dosage of KW-3902 can be increased or decreased either during the treatment or as the initial dose. In addition, the dosage of furosemide can be increased to 60 mg, 80 mg, 100 mg, 120 mg, 140 mg, or 160 mg either during the treatment or as the initial dose

What is claimed is:

1. A method maintaining or restoring the diuretic effect of a non adenosine-modifying diuretic in a subject, comprising:
administering to said subject a composition comprising at least 20 mg KW-3902 or a pharmaceutically acceptable salt, ester prodrug, amide, or metabolite thereof to said subject while maintaining a Cₘₐₓ of the combined KW-3902 and the M1-trans metabolite thereof below about 600 nM following administration.

2. The method of claim 1, wherein said Cₘₐₓ is below about 550 nM following administration.

3. The method of claim 1, wherein said composition comprises at least 30 mg of KW-3902.

4. The method of claim 1, wherein said composition is administered intravenously.

5. The method of claim 2, wherein said composition is administered over about 3.5 to about 4.5 hours.

6. The method of claim 1, wherein said composition is administered orally.

7. The method of claim 2, wherein the Cₘᵢₙ is maintained above about 25 Nm.

8. The method of claim 1, wherein the AUC is about 1000 to about 4000 nM * hr.

9. The method of claim 1, wherein said subject is identified as having a condition selected from the group consisting of: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

10. A method of inducing diuresis in a subject, comprising:
identifying a patient in need of diuresis;
administering a composition comprising at least 20 mg of KW-3902 or a pharmaceutically acceptable salt, ester prodrug, amide, or metabolite thereof to said subject while maintaining a Cₘₐₓ below about 500 nM over a period of about 24 hours following administration; and
administering to said subject a non adenosine modifying diuretic.

11. The method of claim 10, wherein said Cₘₐₓ is below about 550 nM following administration.

12. The method of claim 10, wherein said composition comprises at least 30 mg of KW-3902.

13. The method of claim 10, wherein said composition is administered intravenously.

14. The method of claim 13, wherein said composition is administered over about 3.5 to about 4.5 hours.

15. The method of claim 10, wherein said composition is administered orally.

16. The method of claim 10, wherein the Cₘᵢₙ is maintained above about 25 nM.

17. The method of claim 10, wherein the AUC is about 1000 to about 4000 nM * hr.

18. The method of claim 10, wherein said subject is identified as having a condition selected from the group consisting of: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

19. The method of claim 10, wherein said subject is refractory to standard diuretic therapy.

20. A method for maintaining, restoring or improving renal function in a subject, comprising:
identifying a subject in need thereof; and
administering a composition comprising at least 20 mg of KW-3902 or a pharmaceutically acceptable salt, prodrug, ester, amide, or metabolite thereof to said subject while maintaining a Cₘₐₓ below about 500 nM over a period of about 24 hours following administration.

21. The method of claim 20, wherein said Cₘₐₓ is below about 550 nM following administration.

22. The method of claim 20, wherein said composition comprises at least 30 mg of KW-3902.

23. The method of claim 20, wherein said composition is administered intravenously.

24. The method of claim 23, wherein said composition is administered over about 3.5 to about 4.5 hours.

25. The method of claim 20, wherein said composition is administered orally.

26. The method of claim 20, wherein the Cₘᵢₙ is maintained above about 25 nM.

27. The method of claim 20, wherein the AUC is about 1000 to about 4000 nM * hr.

28. The method of claim 20, wherein said subject is identified as having a condition selected from the group consisting of: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

29. A method of preventing or delaying the onset of renal impairment in a subject with fluid overload or CHF, comprising:
administering a composition comprising at least 20 mg of KW-3902 or a pharmaceutically acceptable salt, ester prodrug, amide, or metabolite thereof to said subject while maintaining a Cₘₐₓ below about 500 nM over a period of about 24 hours following administration.

30. The method of claim 29, wherein said Cₘₐₓ is below about 550 nM following administration.

31. The method of claim 29, wherein said composition comprises at least 30 mg of KW-3902.

32. The method of claim 29, wherein said composition is administered intravenously.

33. The method of claim 32, wherein said composition is administered over about 3.5 to about 4.5 hours.

34. The method of claim 29, wherein said composition is administered orally.

35. The method of claim 29, wherein the Cₘᵢₙ is maintained above about 25 nM.

36. The method of claim 29, wherein the AUC is about 1000 to about 4000 nM * hr.

37. The method of claim 29, wherein said subject is identified as having a condition selected from the group consisting of: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

38. A method of treating a subject suffering from CHF comprising:
administering a composition comprising at least 20 mg of KW-3902 or a pharmaceutically acceptable salt, ester prodrug, amide, or metabolite thereof to said subject while maintaining a Cₘₐₓ below about 500 nM over a period of about 24 hours following administration.

39. The method of claim 38, wherein said Cₘₐₓ is below about 550 nM following administration.

40. The method of claim 38, wherein said composition comprises at least 30 mg of KW-3902.

41. The method of claim 38, wherein said composition is administered intravenously.

42. The method of claim 41, wherein said composition is administered over about 3.5 to about 4.5 hours.

43. The method of claim 38, wherein said composition is administered orally.

44. The method of claim 38, wherein the Cₘᵢₙ is maintained above about 25 nM.

45. The method of claim 38, wherein the AUC is about 1000 to about 4000 nM * hr.

46. The method of claim 38, wherein said subject is identified as having a condition selected from the group consisting of: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, and advanced multiple sclerosis.

47. The method of claim 20, wherein said subject has a decreasing creatinine clearance rate.

48. The method of any one of claims 1, 10, 20, 29, or 38, further comprising:
administering a therapeutically or prophylactically effective amount of an anticonvulsant to said subject.

49. The method of claim 48, wherein said anticonvulsant is selected from the group consisting of diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof.

50. The method of any one of claims 1, 10, 20, 29, or 38, wherein said composition is administered to said subject on a bi-weekly to monthly basis.

51. The method of claim 50, wherein said composition is administered in approximately 14 day intervals.

52. The method of any one of claims 1, 10, 20, 29, or 38, wherein said composition is administered at least once a day.

53. The method of claim 52 wherein said composition is administered between twice a day and four times a day.
